(19) United States
(12) Patent Application Publication
(10) Pub. No.: US 2003/0106085 A1
(43) Pub. Date: Jun. 5, 2003

(54) Disruption of the prostaglandin E synthase 2 gene

(75) Inventors: Laurent P. Audoly, Groton, CT (US); Omar L. Francone, East Lyme, CT (US); Mehrdad Haghpassand, North Stonington, CT (US); John E. Hambor, Madison, CT (US); Marsha L. Roach, Waterford, CT (US); Jeffrey L. Stock, North Stonington, CT (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/293,172

(22) Filed: Nov. 13, 2002

Related U.S. Application Data
(60) Provisional application No. 60/337,431, filed on Nov. 30, 2001.
(60) Provisional application No. 60/405,652, filed on Aug. 22, 2002.

(51) Int. Cl. A01K 67/027; C12N 5/06

(52) U.S. Cl. 800/18; 435/354

(57) ABSTRACT

The present invention features genetically-modified non-human mammals and animal cells containing a disrupted prostaglandin E synthase 2 gene as well as methods of treating an inflammation-mediated disorder involving administering an agent that inhibits prostaglandin E synthase 2.

This application claims priority, under 35 U.S.C. §119(e), from U.S. provisional application No. 60/337,431, filed Nov. 30, 2001, and U.S. provisional application No. 60/405,652, filed Aug. 22, 2002, incorporated herein in their entireties by reference.

### FIELD OF THE INVENTION

The present invention features genetically-modified non-human mammals and animal cells containing a disrupted prostaglandin E synthase 2 gene as well as methods of treating an inflammation-mediated disorder involving administering an agent that inhibits prostaglandin E synthase 2.

### BACKGROUND OF THE INVENTION

Prostaglandin E2 (PGE2) is a major prostanoid derived from prostaglandin H2 (PGH2), either by degradation of PGH2 or by a reaction catalyzed by prostaglandin E synthase (PGES) (Jakobsen et al., Proc. NatI. Acad. Sci. USA 96: 7220-25, 1999). PGH2, formed in a reaction catalyzed by either cyclooxygenase (COX)-1 or COX-2, serves as a precursor to all prostanoid products formed, including prostaglandins, prostacyclin, and thromboxanes (Smith and Marnett, Biochim. Biophys. Acta 1083: 1-17, 1991; Vane and Botting, Inflamm. Res. 44: 1-10, 1995; Herschman, Biochim. Biophys. Acta 1299: 125-40, 1996).

Studies with a specific monoclonal antibody to PGE2 indicate that PGE2 is the major prostanoid contributing to inflammation (Portanova et al., J. Exp. Med. 184: 883-91, 1996). Injection of PGE2 elicits inflammation via vasodilation with plasma extravasation and sensitization of nociceptors (Vane and Botting, Inflamm. Res. 47 (Suppl. 2): S78, 1997). Furthermore, PGE2 stimulates the production of matrix metalloproteinases (Mehindate et al., J. Immunol. 155: 3570, 1995), stimulates angiogenesis (Ben-Av et al., FEBS Lett. 372: 83, 1995), and inhibits T 35 lymphocyte apoptosis (Goetzl et al., J. Immunol. 154: 1041, 1995).

One form of PGES (PGES1 or cPGES) is constitutively expressed in the cytosol of various mammalian cell lines and is generally unaltered by stimulation with bacterial lipopolysaccharide (LPS) (Tanioko et al., J. Biol. Chem. 42: 32775, 2000). An inducible form of PGES (PGES2, iPGES, or mPGES-1) is localized to the microsomal compartment. It is noted that mPGES-1 has become the nomenclature of choice for PGES2. The PGES2 enzyme has been identified as a member of the membrane-associated proteins involved in eicosanoid and glutathione metabolism, and is induced by interleukin (IL)-1β (Jakobsen et al., Proc. Natl. Acad. Sci. USA 96: 7220, 1999; Thoren and Jakobsen, Eur. J. Biochem. 267: 6428, 2000). The enzyme was originally called microsomal glutathione S-transferase 1-like 1 (Jakobsen et al., Protein Sci. 8: 689, 1999).

Various studies have indicated that COX-2 and PGES2 are regulated in a coordinate fashion, and suggest that PGES2 is a key enzyme involved in the formation of PGE2 in COX-2-mediated responses related to inflammation, pyretic effects, and cellular growth regulation (Yamagata et al., J. Neuroscience 21: 2669-77, 2001; Murakami et al., J. Biol. Chem. 275: 32783-92, 2000). Therefore, agents that inhibit PGES2 are suggested to provide therapeutics as an alternative, or in addition to, COX-2 inhibitors (Stichtenoth et al., J. Immunol. 167: 469-74, 2001). However, the complete effects of PGES2 inhibition still remain to be resolved. Thus, there is a need for additional research tools, including PGES2 knockout mice and PGES2 knockout ES cells, to further define the role of PGES2 in inflammatory responses and the therapeutic implications associated with modulating PGES2 activity.

### SUMMARY OF THE INVENTION

The present invention features genetically-modified non-human mammals and animal cells that are homozygous or heterozygous for a disrupted PGES2 gene.

In the first aspect, the invention features a genetically-modified, non-human mammal, wherein the modification results in a disrupted PGES2 gene. Preferably, the mammal is a rodent, more preferably, a mouse, and/or the mammal demonstrates an attenuated response to an experimentally induced model of inflammation, e.g., reduced joint inflammation, reduced white blood cell infiltration, reduced proteoglycan loss at a joint articular surface, and/or reduced inflammatory pain detection. In another preferred embodiment, the mammal further comprises a disrupted ApoE gene.

The second aspect of the invention features a genetically-modified animal cell, wherein the modification comprises a disrupted PGES2 gene. In preferred embodiments, the cell is an embryonic stem (ES) cell, an ES-like cell, or an ES cell-derived macrophage, the cell is cultured in media supplemented with PGE2, and/or the cell is murine or human. In another preferred embodiment, the cell demonstrates reduced PGE2 production under inflammatory conditions. In another preferred embodiment, the cell is isolated from a genetically-modified, non-human mammal containing a modification that results in a disrupted PGES2 gene.

In the third aspect, the invention features a method of identifying a gene that demonstrates modified expression as a result of modified PGES2 activity in an animal cell, said method comprising comparing the expression profile of a genetically modified animal cell, wherein the cell is homozygous for a genetic modification that disrupts the PGES2 gene, to a wild type cell.

The fourth aspect of the invention features a method of treating an inflammation-mediated disorder involving administering an agent that inhibits prostaglandin E synthase 2. Such inflammation includes chronic inflammation (e.g., rheumatoid arthritis and Th1-mediated disorders such as multiple sclerosis), and acute inflammatory pain (e.g., injury-mediated pain). Preferably, the agent is administered in an amount sufficient to reduce joint inflammation, white blood cell infiltration, proteoglycan loss at a joint articular surface, and/or inflammatory pain detection.

Those skilled in the art will fully understand the terms used herein in the description and the appendant claims to describe the present invention. Nonetheless, unless otherwise provided herein, the following terms are as described immediately below.

A non-human mammal or an animal cell that is “genetically-modified” is heterozygous or homozygous for a modification that is introduced into the non-human mammal or animal cell, or into a progenitor non-human mammal or animal cell, by genetic engineering. The standard methods of genetic engineering that are available for introducing the modification include homologous recombination, viral vector gene trapping, irradiation, chemical mutagenesis, and the transgenic expression of a nucleotide sequence encoding antisense RNA alone or in combination with catalytic ribozymes. Preferred methods for genetic modification to disrupt a gene are those, which modify an endogenous gene by inserting a “foreign nucleic acid sequence” into the gene locus, e.g., by homologous recombination or viral vector gene trapping. A “foreign nucleic acid sequence” is an exogenous sequence that is non-naturally occurring in the gene. This insertion of foreign DNA can occur within any region of the PGES2 gene, e.g., in an enhancer, promoter, regulator region, noncoding region, coding region, intron, or exon. The most preferred method of genetic engineering for gene disruption is homologous recombination, in which the foreign nucleic acid sequence is inserted in a targeted manner either alone or in combination with a deletion of a portion of the endogenous gene sequence.

By a PGES2 gene that is “disrupted” is meant a PGES2 gene that is genetically modified such that the cellular activity of the PGES2 polypeptide encoded by the disrupted gene is decreased or eliminated in cells that normally express a wild type version of the PGES2 gene. When the genetic modification effectively eliminates all wild type copies of the PGES2 gene in a cell (e.g., the genetically-modified, non-human mammal or animal cell is homozygous for the PGES2 gene disruption or the only wild type copy of the PGES2 gene originally present is now disrupted), the genetic modification results in a reduction in PGES2 polypeptide activity as compared to a control cell that expresses the wild type PGES2 gene. This reduction in PGES2 polypeptide activity results from either reduced PGES2 gene expression (i.e., PGES2 mRNA levels are effectively reduced resulting in reduced levels of PGES2 polypeptide) and/or because the disrupted PGES2 gene encodes a mutated polypeptide with altered, e.g., reduced, function or stability as compared to a wild type PGES2 polypeptide. Preferably, the activity of PGES2 polypeptide in the genetically-modified, non-human mammal or animal cell is reduced to 50% or less of wild type levels, more preferably, to 25% or less, and, even more preferably, to 10% or less of wild type levels. Most preferably, the PGES2 gene disruption results in non-detectable PGES2 activity as assessed by known methodologies.

By a “genetically-modified, non-human mammal” containing a disrupted PGES2 gene is meant a non-human mammal that is originally produced, for example, by creating a blastocyst or embryo carrying the desired genetic modification and then implanting the blastocyst or embryo in a foster mother for in utero development. The genetically-modified blastocyst or embryo can be made, in the case of mice, by implanting a genetically-modified embryonic stem (ES) cell into a mouse blastocyst or by aggregating ES cells with tetraploid embryos. In another method chimeric animals may be created by aggregation using ES cells and morula stage (8 cell) embryos (diploid). Alternatively, various species of genetically-modified embryos can be obtained by nuclear transfer. In the case of nuclear transfer, the donor cell is a somatic cell or a pluripotent stem cell, and it is engineered to contain the desired genetic modification that disrupts the PGES2 gene. The nucleus of this cell is then transferred into a fertilized or parthenogenetic oocyte that is enucleated; the resultant embryo is reconstituted and developed into a blastocyst. A genetically-modified blastocyst produced by either of the above methods is then implanted into a foster mother according to standard methods well known to those skilled in the art. A “genetically-modified, non-human mammal” includes all progeny of the non-human mammals created by the methods described above, provided that the progeny inherit at least one copy of the genetic modification that disrupts the PGES2 gene. It is preferred that all somatic cells and germlne cells of the genetically-modified non-human mammal contain the modification. Preferred non-human mammals that are genetically-modified to contain a disrupted PGES2 gene include rodents, such as mice and rats, cats, dogs, rabbits, guinea pigs, hamsters, sheep, pigs, and ferrets.

By a “genetically-modified animal cell” containing a disrupted PGES2 gene is meant an animal cell, including a human cell, created by genetic engineering to contain a disrupted PGES2 gene, as well as daughter cells that inherit the disrupted PGES2 gene. These cells may be genetically-modified in culture according to any standard method known in the art. As an alternative to genetically modifying the cells in culture, non-human mammalian cells may also be isolated from a genetically-modified, non-human mammal that contains a PGES2 gene disruption. The animal cells of the invention may be obtained from primary cell or tissue preparations as well as culture-adapted, tumorigenic, or transformed cell lines. These cells and cell lines are derived, for example, from endothelial cells, epithelial cells, islets, neurons and other neural tissue-derived cells, mesothelial cells, osteocytes, lymphocytes, chondrocytes, hematopoietic cells, immune cells, cells of the major glands or organs (e.g., testicle, liver, lung, heart, stomach, pancreas, kidney, and skin), muscle cells (including cells from skeletal muscle, smooth muscle, and cardiac muscle), exocrine or endocrine cells, fibroblasts, and embryonic and other totipotent or pluripotent stem cells (e.g., ES cells, ES-like cells, and embryonic germlne (EG) cells, and other stem cells, such as progenitor cells and tissue-derived stem cells). The preferred genetically-modified cells are ES cells, more preferably, mouse or rat ES cells, and, most preferably, human ES cells.

By an “ES cell” or an “ES-like cell” is meant a pluripotent stem cell derived from an embryo, from a primordial germ cell, or from a teratocarcinoma, that is capable of indefinite self renewal as well as differentiation into cell types that are representative of all three embryonic germ layers.

By “modified PGES2 activity” is meant a change in the activity of the PGES2 enzyme as a result of genetic manipulation of the PGES2 gene that causes a change in the level of functional PGES2 enzyme in the cell, or as the result of administration of a pharmacological agent that agonizes or antagonizes PGES2 activity.

Other features and advantages of the invention will be apparent from the following detailed description and from the claims. While the invention is described in connection with specific embodiments, it will be understood that other changes and modifications that may be practiced are also part of this invention and are also within the scope of the appendant claims. This application is intended to cover any equivalents, variations, uses, or adaptations of the invention that follow, in general, the principles of the invention, including departures from the present disclosure that come within known or customary practice within the art, and that are able to be ascertained without undue experimentation. Additional guidance with respect to making and using nucleic acids and polypeptides is found in standard textbooks of molecular biology, protein science, and immunology (see, e.g., Davis et al., Basic Methods in Molecular Biology, Elsevir Sciences Publishing, Inc., New York, N.Y.,1986; Hames et al., Nucleic Acid Hybridization, IL Press, 1985; Molecular Cloning, Sambrook et al., Current Protocols in Molecular Biology, Eds. Ausubel et al., John Wiley and Sons; Current Protocols in Human Genetics, Eds. Dracopoli et al., John Wiley and Sons; Current Protocols in Protein Science, Eds. John E. Coligan et al., John Wiley and Sons; and Current Protocols in Immunology, Eds. John E. Coligan et al., John Wiley and Sons). All publications mentioned herein are incorporated by reference in their entireties.

### BRIEF DESCRIPTION OF THE FIGURES

FIG. 1 is a schematic depicting an embodiment of a PGES2 gene targeting vector, the location for homologous recombination of the vector in the endogenous murine PGES2 gene, and the positions of primers used to verify gene targeting.

FIG. 2 shows the results of polymerase chain reaction (PCR)-based genotyping of wild-type (+/+), heterozygote (+/−), and knockout (−/−) mice with respect to the disrupted PGES2 allele.

FIG. 3 is a graph showing the effects of a 10 minute stimulation with arachidonic acid (AA) on PGE2 production in ES cell in vitro derived macrophages (ESMs) from PGES2 knockout (−/−), PGES2 heterozygote (+/−), and wild type (+/+) ES cells.

FIG. 4 is a graph showing the effects of stimulation with varying concentrations of lipopolysaccharide (LPS) on PGE2 production in ESMs from PGES2−/−, PGES2+/−, and +/+ ES cells.

FIG. 5 is a graph showing the effects of a 10-minute (10′) stimulation with calcium ionophore A23187 on PGE2 production in ESMs from PGES2−/−, PGES2+/−, and +/+ ES cells.

FIG. 6 is a graph showing the effects of a 10 minute stimulation with arachidonic acid (AA), following a 24 hour simulation with 10 μg/ml LPS, on PGE2 production in ESMs from PGES2 knockout (−/−), PGES2 heterozygote (+/−), and wild type (+/+) ES cells.

FIG. 7 shows arthritic score over time in PGES2−/− and PGES2+/+ collagen immunized male and female mice.

FIG. 8 shows the percent incidence in arthritis over time in PGES2−/− and PGES2+/+ collagen immunized male and female mice.

FIG. 9 shows arthritic score over time in PGES2−/− and PGES2+/+ collagen immunized mice of mixed sex.

FIG. 10 shows present incidence of arthritis overtime in PGES2−/− and PGES2+/+ collagen immunized mice of mixed sex.

FIG. 11 shows the change in paw volume in PGES2+/+ and PGES2-following delayed-type hypersensitivity responses in animals receiving similar immunization protocols.

FIG. 12 shows the number of stretches in time intervals for PGES +/+ and PGES −/− mice treated with piroxicam vs. vehicle.

### DETAILED DESCRIPTION OF THE INVENTION

Genetically-Modified Non-Human Mammals and Animal Cells Containing a Disrupted PGES2 Gene

1. Genetically-Modified Non-Human Mammals and Animal Cells

The genetically-modified, non-human mammals and genetically-modified animal cells, including human cells, of the invention are heterozygous or homozygous for a modification that disrupts the PGES2 gene. The cells may be derived by genetically engineering cells in culture, or, in the case of non-human mammalian cells, the cells may be isolated from genetically-modified, non-human mammals.

The PGES2 gene locus is disrupted by one of the several techniques for genetic modification known in the art, including chemical mutagenesis (Rinchik, Trends in Genetics 7: 15-21, 1991, Russell, Environmental & Molecular Mutagenesis 23 (Suppl. 24): 23-29, 1994), irradiation (Russell, supra), transgenic expression of PGES2 gene antisense RNA, either alone or in combination with a catalytic RNA ribozyme sequence (Luyckx et al., Proc. Natl. Acad. Sci. 96: 12174-79, 1999; Sokol et al., Transgenic Research 5: 363-71, 1996; Efrat et al., Proc. Natl. Acad. Sci. USA 91: 2051-55, 1994; Larsson et al., Nucleic Acids Research 22: 2242-48, 1994) and, as further discussed below, the disruption of the PGES2 gene by the insertion of a foreign nucleic acid sequence into the PGES2 gene locus. Preferably, the foreign sequence is inserted by homologous recombination or by the insertion of a viral vector. Most preferably, the method of PGES2 gene disruption is homologous recombination and includes a deletion of a portion of the endogenous PGES2 gene sequence.

The integration of the foreign sequence disrupts the PGES2 gene through one or more of the following mechanisms: by interfering with the PGES2 gene transcription or translation process (e.g., by interfering with promoter recognition, or by introducing a transcription termination site or a translational stop codon into the PGES2 gene); or by distorting the PGES2 gene coding sequence such that it no longer encodes a PGES2 polypeptide with normal function (e.g., by inserting a foreign coding sequence into the PGES2 gene coding sequence, by introducing a frameshift mutation or amino acid(s) substitution, or, in the case of a double crossover event, by deleting a portion of the PGES2 gene coding sequence that is required for expression of a functional PGES2 protein).

To insert a foreign sequence into a PGES2 gene locus in the genome of a cell to create the genetically modified non-human mammals and animal cells of the invention based upon the present description, the foreign DNA sequence is introduced into the cell according to a standard method known in the art such as electroporation, calcium-phosphate precipitation, retroviral infection, microinjection, biolistics, liposome transfection, DEAE-dextran transfection, or transferrinfection (see, e.g., Neumann et al., EMBO J. 1: 841-845, 1982; Pofter et al., Proc. Natl. Acad. Sci USA 81: 7161-65, 1984; Chu et al., Nucleic Acids Res. 15: 1311-26, 1987; Thomas and Capecchi, Cell 51: 503-12, 1987; Baum et al., Biotechniques 17: 1058-62, 1994; Biewenga et al., J. Neuroscience Methods 71: 67-75, 1997; Zhang et al., Biotechniques 15: 868-72, 1993; Ray and Gage, Biotechniques 13: 598-603, 1992; Lo, Mol. Cell. Biol. 3: 1803-14, 1983; Nickoloff et al., Mol. Biotech. 10: 93-101, 1998; Linney et al., Dev. Biol. (Orlando) 213: 207-16, 1999; Zimmer and Gruss, Nature 338: 150-153, 1989; and Robertson et al., Nature 323: 445-48, 1986). The preferred method for introducing foreign DNA into a cell is electroporation.

2. Homologous Recombination

The method of homologous recombination targets the PGES2 gene for disruption by introducing a PGES2 gene targeting vector into a cell containing a PGES2 gene. The ability of the vector to target the PGES2 gene for disruption stems from using a nucleotide sequence in the vector that is homologous, i.e., related, to the PGES2 gene. This homology region facilitates hybridization between the vector and the endogenous sequence of the PGES2 gene. Upon hybridization, the probability of a crossover event between the targeting vector and genomic sequences greatly increases. This crossover event results in the integration of the vector sequence into the PGES2 gene locus and the functional disruption of the PGES2 gene.

General principles regarding the construction of vectors used for targeting are reviewed in Bradley et al. (Biotechnol. 10: 534, 1992). Two different types of vector can be used to insert DNA by homologous recombination: an insertion vector or a replacement vector. An insertion vector is circular DNA, which contains a region of PGES2 gene homology with a double stranded break. Following hybridization between the homology region and the endogenous PGES2 gene, a single crossover event at the double stranded break results in the insertion of the entire vector sequence into the endogenous gene at the site of crossover.

The more preferred vector to create the genetically modified non-human mammals and animals cells of the invention by homologous recombination is a replacement vector, which is colinear rather than circular. Replacement vector integration into the PGES2 gene requires a double crossover event, i.e. crossing over at two sites of hybridization between the targeting vector and the PGES2 gene. This double crossover event results in the integration of a vector sequence that is sandwiched between the two sites of crossover into the PGES2 gene and the deletion of the corresponding endogenous PGES2 gene sequence that originally spanned between the two sites of crossover (see, e.g., Thomas and Capecchi et al., Cell 51: 503-12, 1987; Mansour et al., Nature 336: 348-52, 1988; Mansour et al., Proc. Natl. Acad. Sci. USA 87: 7688-7692, 1990; and Mansour, GATA 7: 219-227, 1990).

A region of homology in a targeting vector to create the genetically modified non-human mammals and animal cells of the invention is generally at least 100 nucleotides in length. Most preferably, the homology region is at least 1-5 kilobases (kb) in length. Although there is no demonstrated minimum length or minimum degree of relatedness required for a homology region, targeting efficiency for homologous recombination generally corresponds with the length and the degree of relatedness between the targeting vector and the PGES2 gene locus. In the case where a replacement vector is used, and a portion of the endogenous PGES2 gene is deleted upon homologous recombination, an additional consideration is the size of the deleted portion of the endogenous PGES2 gene. If this portion of the endogenous PGES2 gene is greater than 1 kb in length, then a targeting cassette with regions of homology that are longer than 1 kb is recommended to enhance the efficiency of recombination. Further guidance regarding the selection and use of sequences effective for homologous recombination, based on the present description, is described in the literature (see, e.g., Deng and Capecchi, Mol. Cell. Biol. 12: 3365-3371, 1992; Bollag et al., Annu. Rev. Genet. 23: 199-225, 1989; and Waldman and Liskay, Mol. Cell. Biol. 8: 5350-5357, 1988).

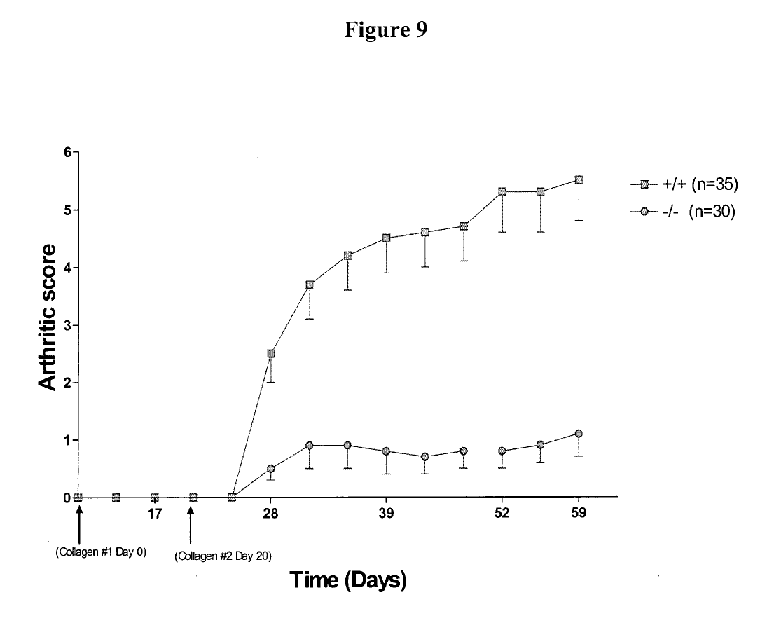

As those skilled in the art will recognize, a wide variety of cloning vectors may be used as vector backbones in the construction of the PGES2 gene targeting vectors of the present invention, including pBluescript-related plasmids (e.g., Bluescript KS+11), pQE70, pQE60, pQE-9, pBS, pD10, phagescript, phiX174, pBK Phagemid, pNH8A, pNH16a, pNH18Z, pNH46A, ptrc99a, pKK223-3, pKK233-3, pDR540, and pRIT5 PWLNEO, pSV2CAT, pXT1, pSG (Stratagene), pSVK3, PBPV, PMSG, and pSVL, pBR322 and pBR322-based vectors, pMB9, pBR325, pKH47, pBR328, pHC79, phage Charon 28, pKBl 1, pKSV-10, pK19 related plasmids, pUC plasmids, and the pGEM series of plasmids. These vectors are available from a variety of commercial sources (e.g., Boehringer Mannheim Biochemicals, Indianapolis, Ind.; Qiagen, Valencia, Calif.; Stratagene, La Jolla, Calif.; Promega, Madison, Wis.; and New England Biolabs, Beverly, Mass.). However, any other vectors, e.g. plasmids, viruses, or parts thereof, may be used as long as they are replicable and viable in the desired host. The vector may also comprise sequences which enable it to replicate in the host whose genome is to be modified. The use of such a vector can expand the interaction period during which recombination can occur, increasing the efficiency of targeting (see Molecular Biology, ed. Ausubel et al, Unit 9.16, Fig. 9.16.1).

The specific host employed for propagating the targeting vectors of the present invention is not critical. Examples include E. coli K12 RR1 (Bolivar et al., Gene 2: 95, 1977), E. coli K112 HB101 (ATCC No. 33694), E. coli MM21 (ATCC No. 336780), E. coli DH1 (ATCC No. 33849), E. coli strain DH5a, and E. coli STBL2. Alternatively, hosts such as C. cerevisiae or B. subtilis can be used. The above-mentioned hosts are available commercially (e.g., Stratagene, La Jolla, Calif.; and Life Technologies, Rockville, Md.).

To create the targeting vector, a PGES2 gene targeting construct is added to an above-described vector backbone. The PGES2 gene targeting constructs of the invention have at least one PGES2 gene homology region. To make the PGES2 gene homology regions, a PGES2 genomic or cDNA sequence is used as a basis for producing polymerase chain reaction (PCR) primers. These primers are used to amplify the desired region of the PGES2 sequence by high fidelity PCR amplification (Mattila et al., Nucleic Acids Res. 19: 4967, 1991; Eckert and Kunkel 1: 17, 1991; and U.S. Pat. No. 4,683,202). The genomic sequence is obtained from a genomic clone library or from a preparation of genomic DNA, preferably from the animal species that is to be targeted for PGES2 gene disruption. The murine PGES2 cDNA sequence can be used in making a PGES2 targeting vector (Genbank NM 022415 and AB041997).

Preferably, the targeting constructs of the invention also include an exogenous nucleotide sequence encoding a positive marker protein. The stable expression of a positive marker after vector integration confers an identifiable characteristic on the cell, ideally, without compromising cell viability. Therefore, in the case of a replacement vector, the marker gene is positioned between two flanking homology regions so that it integrates into the PGES2 gene following the double crossover event in a manner such that the marker gene is positioned for expression after integration. It is preferred that the positive marker protein is a selectable protein; the stable expression of such a protein in a cell confers a selectable phenotypic characteristic, i.e., the characteristic enhances the survival of the cell under otherwise lethal conditions. Thus, by imposing the selectable condition, one can isolate cells that stably express the positive selectable marker-encoding vector sequence from other cells that have not successfully integrated the vector sequence on the basis of viability. Examples of positive selectable marker proteins (and their agents of selection) include neo (G418 or kanomycin), hyg (hygromycin), hisD (histidinol), gpt (xanthine), ble (bleomycin), and hprt (hypoxanthine) (see, e.g., Capecchi and Thomas, U.S. Pat. No. 5,464,764, and Capecchi, Science 244: 1288-92, 1989). Other positive markers that may also be used as an alternative to a selectable marker include reporter proteins such as β-galactosidase, firefly luciferase, or GFP (see, e.g., Current Protocols in Cytometry, Unit 9.5, and Current Protocols in Molecular Biology, Unit 9.6, John Wiley & Sons, New York, N.Y., 2000).

The above-described positive selection step does not distinguish between cells that have integrated the vector by targeted homologous recombination at the PGES2 gene locus versus random, non-homologous integration of vector sequence into any chromosomal position. Therefore, when using a replacement vector for homologous recombination, it is also preferred to include a nucleotide sequence encoding a negative selectable marker protein or a suitable alternate. Examples of negative selectable marker causes a cell expressing the marker to lose viability when exposed to a certain agent (i.e., the marker protein becomes lethal to the cell under certain selectable conditions). Examples of negative selectable markers (and their agents of lethality) include herpes simplex virus thymidine kinase (gancyclovir or 1,2-deoxy-2-fluoro-α-d-arabinofuransyl-5-iodouracil), Hprt (6-thioguanine or 6-thioxanthine), and diphtheria toxin, ricin toxin, and cytosine deaminase (5-fluorocytosine).

The nucleotide sequence encoding the negative selectable marker is positioned outside of the two homology regions of the replacement vector. Given this positioning, cells will only integrate and stably express the negative selectable marker if integration occurs by random, non-homologous recombination; homologous recombination between the PGES2 gene and the two regions of homology in the targeting construct excludes the sequence encoding the negative selectable marker from integration. Thus, by imposing the negative condition, cells that have integrated the targeting vector by random, non-homologous recombination lose viability.

The above-described combination of positive and negative selectable markers is preferred because a series of positive and negative selection steps can be designed to more efficiently select only those cells that have undergone vector integration by homologous recombination, and, therefore, have a potentially disrupted PGES2 gene. Further examples of positive-negative selection schemes, selectable markers, and targeting constructs are described, for example, in U.S. Pat. No. 5,464,764, WO 94/06908, and Valancius and Smithies, Mol. Cell. Biol. 11: 1402, 1991.

For a marker protein to be stably expressed upon vector integration, the targeting vector may be designed so that the marker coding sequence is operably linked to the endogenous PGES2 gene promoter upon vector integration. Expression of the marker is then driven by the PGES2 gene promoter in cells that normally express the PGES2 gene. Alternatively, each marker in the targeting construct of the vector may contain its own promoter that drives expression independent of the PGES2 gene promoter. This latter scheme has the advantage of allowing for expression of markers in cells that do not typically express the PGES2 gene (Smith and Berg, Cold Spring Harbor Symp. Quant. Biol. 49: 171, 1984; Sedivy and Sharp, Proc. Natl. Acad. Sci. (USA) 86: 227, 1989; Thomas and Capecchi, Cell 51: 503, 1987).

Exogenous promoters that can be used to drive marker gene expression include cell-specific or stage-specific promoters, constitutive promoters, and inducible or regulatable promoters. Non-limiting examples of these promoters include the herpes simplex thymidine kinase promoter, cytomegalovirus (CMV) promoter/enhancer, SV40 promoters, PGK promoter, PMC1-neo, metallothionein promoter, adenovirus late promoter, vaccinia virus 7.5K promoter, avian beta globin promoter, histone promoters (e.g., mouse histone H3-614), beta actin promoter, neuron-specific enolase, muscle actin promoter, and the cauliflower mosaic virus 35S promoter (see generally, Sambrook et al., Molecular Cloning, Vols. I-III, Cold Spring Harbor Laboratory Press, Cold Spring Harbor, N.Y., 1989, and Current Protocols in Molecular Biology, John Wiley & Sons, New York, N.Y., 2000); Stratagene, La Jolla, Calif.

To confirm whether cells have integrated the vector sequence into the targeted PGES2 gene locus, primers or genomic probes that are specific for the desired vector integration event can be used in combination with PCR or Southern blot analysis to identify the presence of the desired vector integration into the PGES2 gene locus (Erlich et al., Science 252: 1643-51, 1991; Zimmer and Gruss, Nature 338: 150, 1989;

Mouellic et al., Proc. Natl. Acad. Sci. (USA) 87: 4712, 1990; and Shesely et al., Proc.

Natl. Acad. Sci. (USA) 88: 4294, 1991).

3. Gene Trapping

Another method available for inserting a foreign nucleic acid sequence into the PGES2 gene locus to disrupt the PGES2 gene, based on the present description, is gene trapping. This method takes advantage of the cellular machinery present in all mammalian cells that splices exons into mRNA to insert a gene trap vector coding sequence into a gene in a random fashion. Once inserted, the gene trap vector creates a mutation that may disrupt the trapped PGES2 gene. In contrast to homologous recombination, this system for mutagenesis creates largely random mutations. Thus, to obtain a genetically-modified cell that contains a disrupted PGES2 gene, cells containing this particular mutation must be identified and selected from a pool of cells that contain random mutations in a variety of genes.

Gene trapping systems and vectors have been described for use in genetically modifying murine cells and other cell types (see, e.g., Allen et al., Nature 333: 852-55, 1988; Bellen et al., Genes Dev. 3: 1288-1300, 1989; Bier et al., Genes Dev. 3: 1273-1287, 1989; Bonnerot et al., J. Virol. 66: 4982-91, 1992; Brenner et al., Proc. Nat. Acad. Sci. USA 86: 5517-21, 1989; Chang et al., Virology 193: 73747, 1993; Friedrich and Soriano, Methods Enzymol. 225: 681-701, 1993; Friedrich and Soriano, Genes Dev. 5: 1513-23, 1991; Goff, Methods Enzymol. 152: 469-81, 1987; Gossler et al., Science 244: 463-65, 1989; Hope, Develop. 113: 399408, 1991; Kerr et al., Cold Spring Harb. Symp. Quant. Biol. 2: 767-776, 1989; Reddy et al., J. Virol. 65: 1507-1515, 1991; Reddy et al., Proc. Natl. Acad. Sci. U.S.A. 89: 6721-25, 1992; Skarnes et al., Genes Dev. 6: 903-918, 1992; von Melchner and Ruley, J. Virol. 63: 3227-3233, 1989; and Yoshida et al., Transgen. Res. 4: 277-87, 1995).

Promoter trap, or 5′, vectors contain, in 5′ to 3′ order, a splice acceptor sequence followed by an exon, which is typically characterized by a translation initiation codon and open reading frame and/or an internal ribosome entry site. In general, these promoter trap vectors do not contain promoters or operably linked splice donor sequences. Consequently, after integration into the cellular genome of the host cell, the promoter trap vector sequence intercepts the normal splicing of the upstream gene and acts as a terminal exon. Expression of the vector coding sequence is dependent upon the vector integrating into an intron of the disrupted gene in the proper reading frame. In such a case, the cellular splicing machinery splices exons from the trapped gene upstream of the vector coding sequence (Zambrowicz et al., WO 99/50426, U.S. Pat. No. 6,080,576).

An alternative method for producing an effect similar to the above-described promoter trap vector is a vector that incorporates a nested set of stop codons present in, or otherwise engineered into, the region between the splice acceptor of the promoter trap vector and the translation initiation codon or polyadenylation sequence. The coding sequence can also be engineered to contain an independent ribosome entry site (IRES) so that the coding sequence will be expressed in a manner largely independent of the site of integration within the host cell genome. Typically, but not necessarily, an IRES is used in conjunction with a nested set of stop codons.

Another type of gene trapping scheme uses a 3′ gene trap vector. This type of vector contains, in operative combination, a promoter region, which mediates expression of an adjoining coding sequence, the coding sequence, and a splice donor sequence that defines the 3′ end of the coding sequence exon. After integration into a host cell genome, the transcript expressed by the vector promoter is spliced to a splice acceptor sequence from the trapped gene that is located downstream of the integrated gene trap vector sequence. Thus, the integration of the vector results in the expression of a fusion transcript comprising the coding sequence of the 3′ gene trap cassette and any downstream cellular exons, including the terminal exon and its polyadenylation signal. When such vectors integrate into a gene, the cellular splicing machinery splices the vector coding sequence upstream of the 3′ exons of the trapped gene. One advantage of such vectors is that the expression of the 3′ gene trap vectors is driven by a promoter within the gene trap cassette and does not require integration into a gene that is normally expressed in the host cell (Zambrowicz et al., WO 99/50426). Examples of transcriptional promoters and enhancers that may be incorporated into the 3′ gene trap vector include those discussed above with respect to targeting vectors.

The viral vector backbone used as the structural component for the promoter or 3′ gene trap vector may be selected from a wide range of vectors that can be inserted into the genome of a target cell. Suitable backbone vectors include, but are not limited to, herpes simplex virus vectors, adenovirus vectors, adeno-associated virus vectors, retroviral vectors, lentiviral vectors, pseudorabies virus, alpha-herpes virus vectors, and the like. A thorough review of viral vectors, in particular, viral vectors suitable for modifying nonreplicating cells and how to use such vectors in conjunction with the expression of an exogenous polynucleotide sequence, can be found in Viral Vectors: Gene Therapy and Neuroscience Applications, Eds. Caplitt and Loewy, Academic Press, San Diego, 1995.

Preferably, retroviral vectors are used for gene trapping. These vectors can be used in conjunction with retroviral packaging cell lines such as those described in U.S. Pat. No. 5,449,614. Where non-murine mammalian cells are used as target cells for genetic modification, amphotropic or pantropic packaging cell lines can be used to package suitable vectors (Ory et al., Proc. Natl. Acad. Sci., USA 93: 11400-11406, 1996). Representative retroviral vectors that can be adapted to create the presently described 3′ gene trap vectors are described, for example, in U.S. Pat. No. 5,521,076.

The gene trapping vectors may contain one or more of the positive marker genes discussed above with respect to targeting vectors used for homologous recombination. Similar to their use in targeting vectors, these positive markers are used in gene trapping vectors to identify and select cells that have integrated the vector into the cell genome. The marker gene may be engineered to contain an independent ribosome entry site (IRES) so that the marker will be expressed in a manner largely independent of the location in which the vector has integrated into the target cell genome.

Given that gene trap vectors will integrate into the genome of infected host cells in a fairly random manner, a genetically-modified cell having a disrupted PGES2 gene must be identified from a population of cells that have undergone random vector integration. Preferably, the genetic modifications in the population of cells are of sufficient randomness and frequency such that the population represents mutations in essentially every gene found in the cell's genome, making it likely that a cell with a disrupted PGES2 gene will be identified from the population (see Zambrowicz et al.; WO 99/50426; Sands et al., WO 98/14614; U.S. Pat. No. 6,080,576).

Individual mutant cell lines containing a disrupted PGES2 gene are identified in a population of mutated cells using, for example, reverse transcription and PCR to identify a mutation in a PGES2 gene sequence. This process can be streamlined by pooling clones. For example, to find an individual clone containing a disrupted PGES2 gene, RT-PCR is performed using one primer anchored in the gene trap vector and the other primer located in the PGES2 gene sequence. A positive RT-PCR result indicates that the vector sequence is encoded in the PGES2 gene transcript, indicating that PGES2 gene has been disrupted by a gene trap integration event (see, e.g., Sands et al., WO 98/14614, U.S. Pat. No. 6,080,576).

4. Temporal, Spatial, and Inducible PGES2 Gene Disruptions

In certain embodiments of the present invention, a functional disruption of the endogenous PGES2 gene occurs at specific developmental or cell cycle stages (temporal disruption) or in specific cell types (spatial disruption). In other embodiments, the PGES2 gene disruption is inducible when certain conditions are present. A recombinase excision system, such as a Cre-Lox system, may be used to activate or inactivate the PGES2 gene at a specific developmental stage, in a particular tissue or cell type, or under particular environmental conditions. Generally, methods utilizing Cre-Lox technology are carried out, for example, as described by Torres and Kuhn, Laboratory Protocols for Conditional Gene Targeting, Oxford University Press, 1997. Methodology similar to that described for the Cre-Lox system can also be employed utilizing the FLP-FRT system. Further guidance regarding the use of recombinase excision systems for conditionally disrupting genes by homologous recombination or viral insertion is provided, for example, in U.S. Pat. No. 5,626,159, U.S. Pat. No. 5,527,695, U.S. Pat. No. 5,434,066, WO 98/29533, Orban et al., Proc. Nat. Acad. Sci. USA 89: 6861-65, 1992; O'Gorman et al., Science 251: 1351-55, 1991; Sauer et al., Nucleic Acids Research 17: 147-61, 1989; Barinaga, Science 265: 26-28, 1994; and Akagi et al., Nucleic Acids Res. 25: 1766-73, 1997. As those skilled in the art will appreciate, more than one recombinase system can be used to genetically modify a non-human mammal or animal cell.

When using homologous recombination to disrupt the PGES2 gene in a temporal, spatial, or inducible fashion, using a recombinase system such as the Cre-Lox system, a portion of the PGES2 gene coding region is replaced by a targeting construct comprising the PGES2 gene coding region flanked by loxP sites. Non-human mammals and animal cells carrying this genetic modification contain a functional, loxP-flanked PGES2 gene. The temporal, spatial, or inducible aspect of the PGES2 gene disruption is caused by the expression pattern of an additional transgene, a Cre recombinase transgene, that is expressed in the non-human mammal or animal cell under the control of the desired spatially-regulated, temporally-regulated, or inducible promoter, respectively. A Cre recombinase targets the loxP sites for recombination. Therefore, when Cre expression is activated, the LoxP sites undergo recombination to excise the sandwiched PGES2 gene coding sequence, resulting in a functional disruption of the PGES2 gene (Rajewski et al., J. Clin. Invest. 98: 600-03,1996; St.-Onge et al., Nucleic Acids Res. 24: 3875-77, 1996; Agah et al., J. Clin. Invest. 100: 169-79, 1997; Brocard et al., Proc. Natl. Acad. Sci. USA 94: 14559-63, 1997; Feil et al., Proc. Natl. Acad. Sci. USA 93: 10887-90, 1996; and Kuhn et al., Science 269: 1427-29, 1995).

A cell containing both a Cre recombinase transgene and loxP-flanked PGES2 gene can be generated through standard transgenic techniques or, in the case of genetically-modified, non-human mammals, by crossing genetically-modified, non-human mammals wherein one parent contains a loxP flanked PGES2 gene and the other contains a Cre recombinase transgene under the control of the desired promoter. Further guidance regarding the use of recombinase systems and specific promoters to temporally, spatially, or conditionally disrupt the PGES2 gene is found, for example, in Sauer, Meth. Enz. 225: 890-900, 1993, Gu et al., Science 265: 103-06, 1994, Araki et al., J. Biochem. 122: 977-82, 1997, Dymecki, Proc. Natl. Acad. Sci. 93: 6191-96, 1996, and Meyers et al., Nature Genetics 18: 136-41, 1998.

An inducible disruption of the PGES2 gene can also be achieved by using a tetracycline responsive binary system (Gossen and Bujard, Proc. Natl. Acad. Sci. USA 89: 5547-51, 1992). This system involves genetically modifying a cell to introduce a Tet promoter into the endogenous PGES2 gene regulatory element and a transgene expressing a tetracycline-controllable repressor (TetR). In such a cell, the administration of tetracycline activates the TetR which, in turn, inhibits PGES2 gene expression and, therefore, disrupts the PGES2 gene (St.-Onge et al., Nucleic Acids Res. 24: 3875-77, 1996, U.S. Pat. No. 5,922,927).

The above-described systems for temporal, spatial, and inducible disruptions of the PGES2 gene can also be adopted when using gene trapping as the method of genetic modification, for example, as described, in WO 98/29533 and U.S. Pat. No. 6,288,639.

5. Creating Genetically-Modified, Non-human Mammals and Animal Cells

The above-described methods for genetic modification can be used to disrupt a PGES2 gene in virtually any type of somatic or stem cell derived from an animal. Genetically-modified animal cells of the invention include, but are not limited to, mammalian cells, including human cells, and avian cells. These cells may be derived from genetically engineering any animal cell line, such as culture-adapted, tumorigenic, or transformed cell lines, or they may be isolated from a genetically-modified, non-human mammal carrying the desired PGES2 genetic modification.

The cells may be heterozygous or homozygous for the disrupted PGES2 gene. To obtain cells that are homozygous for the PGES2 gene disruption (PGES2−/−), direct, sequential targeting of both alleles can be performed. This process can be facilitated by recycling a positive selectable marker. According to this scheme the nucleotide sequence encoding the positive selectable marker is removed following the disruption of one allele using the Cre-Lox P system. Thus, the same vector can be used in a subsequent round of targeting to disrupt the second PGES2 gene allele (Abuin and Bradley, Mol. Cell. Biol. 16:1851-56,1996; Sedivy et al., T.I.G. 15: 88-90, 1999; Cruz et al., Proc. Natl. Acad. Sci. (USA) 88: 7170-74, 1991; Mortensen et al., Proc. Natl. Acad. Sci. (USA) 88: 7036-40, 1991; Riele et al., Nature (London) 348: 649-651, 1990).

An alternative strategy for obtaining ES cells that are PGES2−/− is the homogenotization of cells from a population of cells that is heterozygous for the PGES2 gene disruption (PGES2+/−). The method uses a scheme in which PGES2+/− targeted clones that express a selectable drug resistance marker are selected against a very high drug concentration; this selection favors cells that express two copies of the sequence encoding the drug resistance marker and are, therefore, homozygous for the PGES2 gene disruption (Mortensen et al., Mol. Cell. Biol. 12: 2391-95, 1992). In addition, genetically-modified animal cells can be obtained from genetically-modified PGES2−/− non-human mammals that are created by mating non-human mammals that are PGES2+/− in germline cells, as further discussed below.

Following the genetic modification of the desired cell or cell line, the PGES2 gene locus can be confirmed as the site of modification by PCR analysis according to standard PCR or Southern blotting methods known in the art (see, e.g., U.S. Pat. No. 4,683,202; and Erlich et al., Science 252: 1643, 1991). Further verification of the functional disruption of the PGES2 gene may also be made if PGES2 gene messenger RNA (mRNA) levels and/or PGES2 polypeptide levels are reduced in cells that normally express the PGES2 gene. Measures of PGES2 gene mRNA levels may be obtained by using reverse transcriptase mediated polymerase chain reaction (RT-PCR), Northern blot analysis, or in situ hybridization. The quantification of PGES2 polypeptide levels produced by the cells can be made, for example, by standard immunoassay methods known in the art. Such immunoassays include, but are not limited to, competitive and non-competitive assay systems using techniques such as RIAs (radioimmunoassays), ELISAs (enzyme-linked immunosorbent assays), “sandwich” immunoassays, immunoradiometric assays, gel diffusion precipitin reactions, immunodiffusion assays, in situ immunoassays (using, for example, colloidal gold, enzymatic, or radioisotope labels), Western blots, 2-dimensional gel analysis, precipitation reactions, immunofluorescence assays, protein A assays, and immunoelectrophoresis assays.

Preferred genetically-modified animal cells are embryonic stem (ES) cells and ES-like cells. These cells are derived from the preimplantation embryos and blastocysts of various species, such as mice (Evans et al., Nature 129:154-156, 1981; Martin, Proc. Natl. Acad. Sci., USA, 78: 7634-7638, 1981), pigs and sheep (Notanianni et al., J. Reprod. Fert. Suppl., 43: 255-260, 1991; Campbell et al., Nature 380: 64-68,1996) and primates, including humans (Thomson et al., U.S. Pat. No. 5,843,780, Thomson et al., Science 282: 1145-1147, 1995; and Thomson et al., Proc. Nati. Acad. Sci. USA 92: 7844-7848, 1995).

These types of ES cells are pluripotent. That is, under proper conditions, they differentiate into a wide variety of cell types derived from all three embryonic germ layers: ectoderm, mesoderm and endoderm. Depending upon the culture conditions, a sample of ES cells can be cultured indefinitely as stem cells, allowed to differentiate into a wide variety of different cell types within a single sample, or directed to differentiate into a specific cell type, such as macrophage-like cells, neuronal cells, cardiomyocytes, chondrocytes, adipocytes, smooth muscle cells, endothelial cells, skeletal muscle cells, keratinocytes, and hematopoietic cells, such as eosinophils, mast cells, erythroid progenitor cells, or megakaryocytes. Directed differentiation is accomplished by including specific growth factors or matrix components in the culture conditions, as further described, for example, in Keller et al., Curr. Opin. Cell Biol. 7: 862-69, 1995, Li et al., Curr. Biol. 8: 971, 1998, Klug et al., J. Clin. Invest. 98: 216-24, 1996, Lieschke et al., Exp. Hematol. 23: 328-34, 1995, Yamane et al., Blood 90: 3516-23, 1997, and Hirashima et al., Blood 93: 1253-63, 1999.

The particular embryonic stem cell line that is used for genetic modification is not critical; exemplary murine ES cell lines include AB-1 (McMahon and Bradley, Cell 62:1073-85, 1990), E14 (Hooper et al., Nature 326: 292-95, 1987), D3 (Doetschman et al., J. Embryol. Exp. Morph. 87: 27-45,1985), CCE (Robertson et al, Nature 323: 445-48, 1986), RW4 (Genome Systems, St. Louis, Mo.), and DBA/1 lacj (Roach et al., Exp. Cell Res. 221: 520-25, 1995). Genetically-modified murine ES cells may be used to generate genetically-modified mice, according to published procedures (Robertson, 1987, Teratocarcinomas and Embryonic Stem Cells: A Practical Approach, Ed. E. J. Robertson, Oxford: IRL Press, pp. 71-112, 1987; Zjilstra et al., Nature 342: 435-438, 1989; and Schwartzberg et al., Science 246: 799-803, 1989).

Following confirmation that the ES cells contain the desired functional disruption of the PGES2 gene, these ES cells are then injected into suitable blastocyst hosts for generation of chimeric mice according to methods known in the art (Capecchi, Trends Genet. 5: 70, 1989). The particular mouse blastocysts employed in the present invention are not critical. Examples of such blastocysts include those derived from C57BL6 mice, C57BL6 Albino mice, Swiss Webster outbred mice, CFLP mice, and MFI mice. Swiss Webster mice are commercially available i.e. CD-1 mice for Charles River Laboratories. Alternatively ES cells may be sandwiched between tetraploid embryos in aggregation wells (Nagy et al., Proc. Natl. Acad. Sci. USA90: 8424-8428, 1993).

The blastocysts or embryos containing the genetically-modified ES cells are then implanted in pseudopregnant female mice and allowed to develop in utero (Hogan et al., Manipulating the Mouse Embryo: A Laboratory Manual, Cold Spring Harbor Laboratory press, Cold Spring Harbor, N.Y. 1988; and Teratocarcinomas and Embryonic Stem Cells: A Practical Approach, E. J. Robertson, ed., IRL Press, Washington, D.C., 1987). The offspring born to the foster mothers may be screened to identify those that are chimeric for the PGES2 gene disruption. Generally, such offspring contain some cells that are derived from the genetically-modified donor ES cell as well as other cells derived from the original blastocyst. In such circumstances, offspring may be screened initially for mosaic coat color, where a coat color selection strategy has been employed, to distinguish cells derived from the donor ES cell from the other cells of the blastocyst. Alternatively, DNA from tail tissue of the offspring can be used to identify mice containing the genetically-modified cells.

The mating of chimeric mice that contain the PGES2 gene disruption in germ line cells produces progeny that possess the PGES2 gene disruption in all germ line cells and somatic cells. Mice that are heterozygous for the PGES2 gene disruption can then be crossed to produce homozygotes (see, e.g., U.S. Pat. No. 5,557,032, and U.S. Pat. No. 5,532,158).

An alternative to the above-described ES cell technology for transferring a genetic modification from a cell to a whole animal is to use nuclear transfer. This method can be employed to make other genetically-modified, non-human mammals besides mice, for example, sheep (McCreath et al., Nature 29: 1066-69, 2000; Campbell et al., Nature 389: 64-66, 1996; and Schnieke et al., Science 278: 2130-33, 1997) and calves (Cibelli et al., Science 280: 1256-58, 1998). Briefly, somatic cells (e.g., fibroblasts) or pluripotent stem cells (e.g., ES-like cells) are selected as nuclear donors and are genetically-modified to contain a functional disruption of the PGES2 gene. When inserting a DNA vector into a somatic cell to mutate the PGES2 gene, it is preferred that a promoterless marker be used in the vector such that vector integration into the PGES2 gene results in expression of the marker under the control of the PGES2 gene promoter (Sedivy and Dutriaux, T.I.G. 15: 88-90, 1999; McCreath et al., Nature 29: 1066-69, 2000). Nuclei from donor cells which have the appropriate PGES2 gene disruption are then transferred to fertilized or parthenogenetic oocytes that are enucleated (Campbell et al., Nature 380: 64, 1996; Wilmut et al., Nature 385: 810, 1997). Embryos are reconstructed, cultured to develop into the morula/blastocyst stage, and transferred into foster mothers for full term in utero development.

The present invention also encompasses the progeny of the genetically-modified, non-human mammals and genetically-modified animal cells. While the progeny are heterozygous or homozygous for the genetic modification that disrupts the PGES2 gene, they may not be genetically identical to the parent non-human mammals and animal cells due to mutations or environmental influences, besides that of the original genetic disruption of the PGES2 gene, that may occur in succeeding generations.

The cells from a non-human genetically modified animal can be isolated from tissue or organs using techniques known to those of skill in the art. In one embodiment, the genetically modified cells of the invention are immortalized. In accordance with this embodiment, cells can be immortalized by genetically engineering the telomerase gene, an oncogene, e.g., mos or v-src, or an apoptosis-inhibiting gene, e.g., bcl-2, into the cells. Alternatively, cells can be immortalized by fusion with a hybridization partner utilizing techniques known to one of skill in the art.

6. “Humanized” Non-Human Mammals and Animal Cells

The genetically-modified non-human mammals and in the case of non-human animal cells of the invention containing a disrupted endogenous PGES2 gene can be further modified to express the human PGES2 sequence (referred to herein as “humanized”). A preferred method for humanizing cells involves replacing the endogenous PGES2 sequence with nucleic acid sequence encoding the human PGES2 sequence (Jakobsson et al., Proc. Natl. Acad. Sci. USA 96: 7220-25, 1999) by homologous recombination. The vectors are similar to those traditionally used as targeting vectors with respect to the 5′ and 3′ homology arms and positive/negative selection schemes. However, the vectors also include sequence that, after recombination, either substitutes the human PGES2 coding sequence for the endogenous sequence, or effects base pair changes, exon substitutions, or codon substitutions that modify the endogenous sequence to encode the human PGES2.

Once homologous recombinants have been identified, it is possible to excise any selection-based sequences (e.g., neo) by using Cre or Flp-mediated site directed recombination (Dymecki, Proc. Natl. Acad. Sci. 93: 6191-96,1996). When substituting the human PGES2 sequence for the endogenous sequence, it is preferred that these changes are introduced directly downstream of the endogenous translation start site. This positioning preserves the endogenous temporal and spatial expression patterns of the PGES2 gene. The human sequence can be the full length human cDNA sequence with a polyA tail attached at the 3′ end for proper processing or the whole genomic sequence (Shiao et al., Transgenic Res. 8: 295-302, 1999). Further guidance regarding these methods of genetically modifying cells and non-human mammals to replace expression of an endogenous gene with its human counterpart is found, for example, in Sullivan et al., J. Biol. Chem. 272: 17972-80, 1997, Reaume et al., J. Biol. Chem. 271: 23380-88, 1996, and Scott et al., U.S. Pat. No. 5,777,194).

Another method for creating such “humanized” organisms is a two step process involving the disruption of the endogenous gene followed by the introduction of a transgene encoding the human sequence by pronuclear microinjection into the knock-out embryos.

7. Uses for the Genetically-Modified Non-Human Mammals and Animal Cells

PGES2 function and therapeutic relevance can be elucidated by investigating the phenotype of the non-human mammals and animals cells of the invention that are homozygous (−/−) and heterozygous (+/−) for the disruption of the PGES2 gene. For example, the genetically-modified PGES2−/− non-human mammals and animal cells can be used to determine whether the PGES2 plays a role in causing, reducing or preventing symptoms or phenotypes to develop in certain models of disease, e.g., arthritis or cancer. If a symptom or phenotype is different in a PGES2−/− non-human mammal or animal cell as compared to a wild type (PGES2+/+) or PGES2+/− non-human mammal or animal cell, then the PGES2 polypeptide plays a role in regulating functions associated with the symptom or phenotype. Examples of animal models that can be used to assess PGES2 function include models to examine chronic and acute inflammatory responses and nociceptive function (e.g., collagen induced arthritis, the air pouch model for white blood cell chemotaxis, carrageenan induced edema, and acetic acid-induced writhing), models to assess cardiorenal function (e.g., measuring urinary prostaglandin excretion as a function of sodium intake), and models to assess thrombosis (e.g., measuring bleeding time and platelet aggregation).

In addition, under circumstances in which an agent has been identified as a PGES2 agonist or antagonist (e.g., the agent significantly modifies one or more of the PGES2 polypeptide activities when the agent is administered to a PGES2+/+ or PGES2+/− non-human mammal or animal cell), the genetically-modified PGES2−/− animal cells of the invention are useful to characterize any other effects caused by the agent besides those known to result from the (ant)agonism of PGES2 (i.e., the non-human mammals and animal cells can be used as negative controls). For example, if the administration of the agent causes an effect in a PGES2+/+ non-human mammal or animal cell that is not known to be associated with PGES2 polypeptide activity, then one can determine whether the agent exerts this effect solely or primarily through modulation of PGES2 by administering the agent to a corresponding PGES2−/− non-human mammal or animal cell. If this effect is absent, or is significantly reduced, in the PGES2−/− non-human mammal or animal cell, then the effect is mediated, at least in part, by PGES2. However, if the PGES2−/− non-human mammal or animal cell exhibits the effect to a degree comparable to the PGES2+/+ or PGES2+/− non-human mammal or animal cell, then the effect is mediated by a pathway that does not involve PGES2 signaling.

Furthermore, if an agent is suspected of possibly exerting an effect via a PGES2 pathway, then the PGES2−/− non-human mammals are useful as negative controls to test this hypothesis. If the agent is indeed acting through PGES2, then the PGES2−/− non-human animals, upon administration of the agent, should not demonstrate a similar effect observed in the PGES2+/+ non-human mammals.

The genetically modified non-human mammals and animal cells of the invention can also be used to identify genes whose expression is upregulated in PGES2+/− or PGES2−/− non-human mammals or animal cells relative to their respective wild-type control. Techniques known to those of skill in the art can be used to identify such genes based upon the present description. For example, DNA assays can be used to identify genes whose expression is upregulated in PGES2+/− or PGES2−/− mice to compensate for a deficiency in PGES2 expression. DNA arrays are known to those of skill in the art and may be sourced commercially, e.g. Affymetrix (see, e.g., U.S. Pat. No. 5,965,352; Schena et al., Science 270:467-470, 1995; DeRisi et al., Nature Genetics 14:457460, 1996; Shalon et al., Genome Res. 6:639-645, 1996; and Schena et al., Proc. Natl. Acad. Sci. (USA) 93:10539-11286, 1995).

### EXAMPLES

A. Library Hybridization

A 335 nucleotide partial cDNA fragment (nucleotides 35-369 of the murine PGES2 cDNA sequence of Genbank ABO41997) was used to hybridize a DBA/1lacJ genomic lambda phage library (Stratagene). Three overlapping PGES2 genomic clones were isolated and subcloned into the Not I site of pBluescript SK+ (Stratagene). These clones were restriction mapped and determined to contain 24 kb of the PGES2 genomic locus, including all 3 exons.

B. Targeting Vector Construction

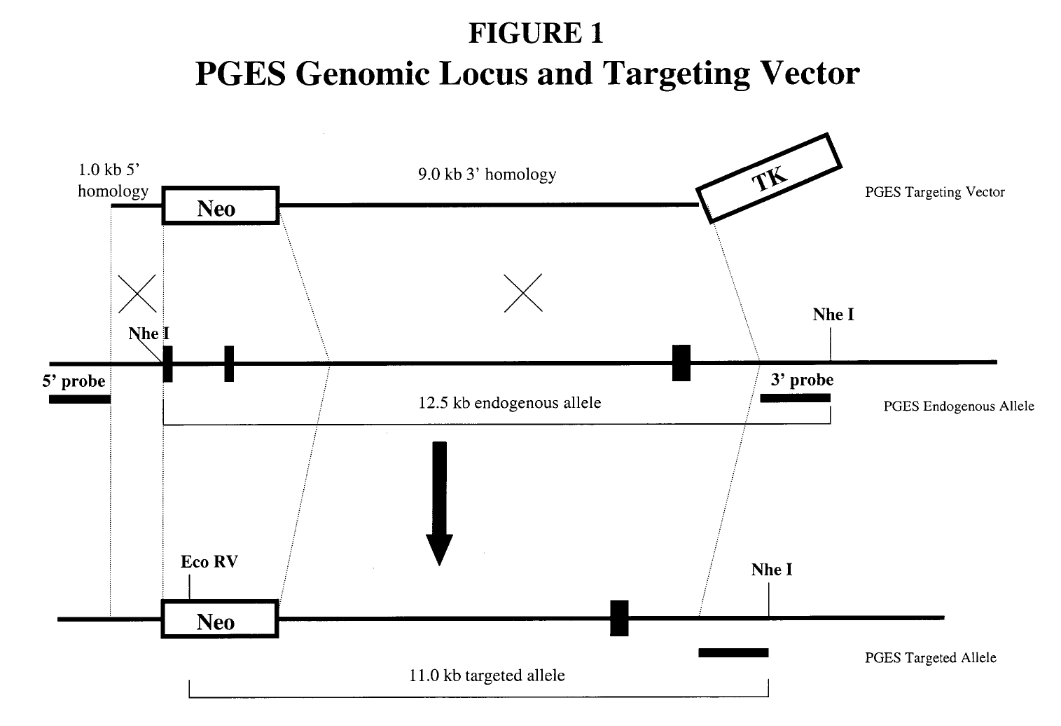

A 1.0 kb Nhe I/Bgl II fragment was isolated from PGES2 genomic clone #24.2-8 and subcloned into the Xba I/BamHI sites of the Litmus 28 vector (New England Biolabs, Beverly, Mass.). This 1.0 kb fragment was re-isolated from the Litmus vector with a Kpn I/Eco RI digestion and cloned into the Kpn I/Eco RI sites of pJNS2-Frt targeting vector backbone (Dombrowicz et al., Cell 75: 969-76, 1993) to serve as the 5′ homology arm. This intermediate clone was designated as PGES2 5′-JNS2Frt clone #10. The 3′ homology arm was also isolated from genomic clone #24.2-8 as a 8.8 kb Not I/Sph I restriction fragment. This 8.8 kb fragment was subcloned into the Eag I/Sph I sites of the cloning vector Litmus 39 (New England Biolabs), and re-isolated from the Litmus 39 vector as an 8.8 kb Sal I fragment. This Sal I fragment was cloned into the Xho site of the PGES2 5′-JNS2Frt clone #10. The final targeting vector clone containing both homology arms, named PGES2-KO clone #5 was designed to replace 3.0 kb of the PGES2 genomic locus with the PGK-neomycin cassette. The 3.0 kb deleted fragment contained part of exon 1 and the entire exon 2, encoding nucleotides 35-256 of the cDNA sequence of Genbank AB041997 (FIG. 1).

C. ES Cell Screening

The PGES2-KO clone #5 targeting vector was linearized with NotI and electroporated into DBA/1LacJ ES cells (Roach et al., Exp. Cell. Res. 221: 520-25, 1995). Pluripotent ES cells were maintained in culture on a Mitomycin C (Sigma Chemical, St. Louis, Mo.) treated primary embryonic fibroblast (PEF) feeder layer in stem cell medium (SCML) which consisted of knockout DMEM (Invitrogen Life Technologies, Inc. (ILTI), Carlsbad, Calif., #10829-018) supplemented with 15% ES cell qualified fetal calf serum (ILTI, #10439-024), 0.1 mM 2-mercaptoethanol (Sigma Chemical, #M-7522), 0.2 mM L-glutamine (ILTI, #25030-081), 0.1 mM MEM non-essential amino acids (ILTI, #11140-050), 1000 units/ml recombinant murine leukemia inhibitory factor (Chemicon International Inc., Temecula, Calif., #ESG-1107) and penicillin/streptomycin (ILTI, #15140-122).

Electroporation of 1×10⁷ cells in SCML and 25 μg linearized targeting vector was carried out using a BTX Electro Cell Manipulator 600 (BTX, Inc., San Diego, Calif.) at a voltage of 240 V, a capacitance of 50 μF and a resistance of 360 Ohms. Positive/negative selection began 24 hours after electroporation in SCML which contained 200 μg/ml G418 (ILTI, #11811-031) and 2 μM gancyclovir (Syntex, Palo Alto, Calif.). Resistant colonies were picked with a micropipette following 8-12 days of selection. Expansion and screening of resistant ES cell colonies were performed as described in Mohn and Koller (Mohn, DNA Cloning 4 (ed. Hames), 143-184, Oxford University Press, New York, 1995).

DNA was isolated from 79 ES cell clones which survived G418 and gancyclovir selection and digested with Nhe I and Eco RV restriction enzymes. The digests were electrophoresed on 0.7% agarose gels (BioWhittaker Molecular Applications, Rockland, Me.) and transferred to Hybond N+ (Amersham Pharmacia Biotech, Buckinghamshire, England) nylon membrane for Southern analysis. A 1.1 kb Kpn I/Nhe I genomic fragment, 3′ of the 8.8 kb 3′ homology arm, was used as a probe to screen for homologous recombination on the 3′ side. The 1.1 kb probe recognizes a 12.5 kb endogenous Nhe I fragment and an 11.0 kb fragment for a targeted allele because of an introduced Eco RV site in the neomycin cassette. From the 79 clones screened, 4 targeted clones were identified with the 1.1 kb 3′ probe (clones #22, #70, #78, and #84). Recombination on the 5′ side was confirmed for these 4 clones using a 400 bp Not I/Bgl II fragment which was 5′ of the 1.0 kb 5′ homology arm. This 400 bp probe recognizes a 12.0 kb endogenous Spe I/Eco RV fragment and a 3.5 kb fragment for a targeted allele because of a new Eco RV site introduced with the neomycin cassette.

D. Knockout Mouse Production and Genotyping

ES cells from targeted clones #22 and #70 were microinjected into blastocysts stage embryos isolated from C57BU6J females (The Jackson Laboratory, Bar Harbor, Me.).

Male chimeras for both clones were identified and back-crossed to DBA/1lacJ females (The Jackson Laboratory) to generate germline PGES2 heterozygous (+/−) offspring. Heterozygous animals were genotyped by PCR for the presence of the neomycin gene using the primer set of Neo-833F(5′ gcaggatctcctgtcatctcacc 3′) (SEQ ID NO: 1) and Neo-1023R (5′ gatgctcttcgtccagatcatcc 3′) (SEQ ID NO: 2). This primer set amplifies a 190 bp fragment from a PGES2 targeted allele.

Homozygous PGES2 knockout mice (PGES2 KO) were generated from heterozygous matings (heterozygous×heterozygous) with normal Mendelian ratios observed. Animals from the heterozygous matings were genotyped by PCR using two oligo sets, one set specific for the targeted allele (Neo PCR) and the other set specific for the PGES2 allele (within the knockout region). The Neo PCR uses the same primer set as used for the heterozygote screening. The oligos for the PGES2 PCR were PGES2KO409F (5′ tcccaggtgttgggatttagacg 3′) (SEQ ID NO: 3) and PGES2KO-821R (5′ taggtggctgtactgtttgttgc 3′) (SEQ ID NO: 4). These oligos amplify a 412 bp fragment and are contained within the 3.0 kb knockout region.

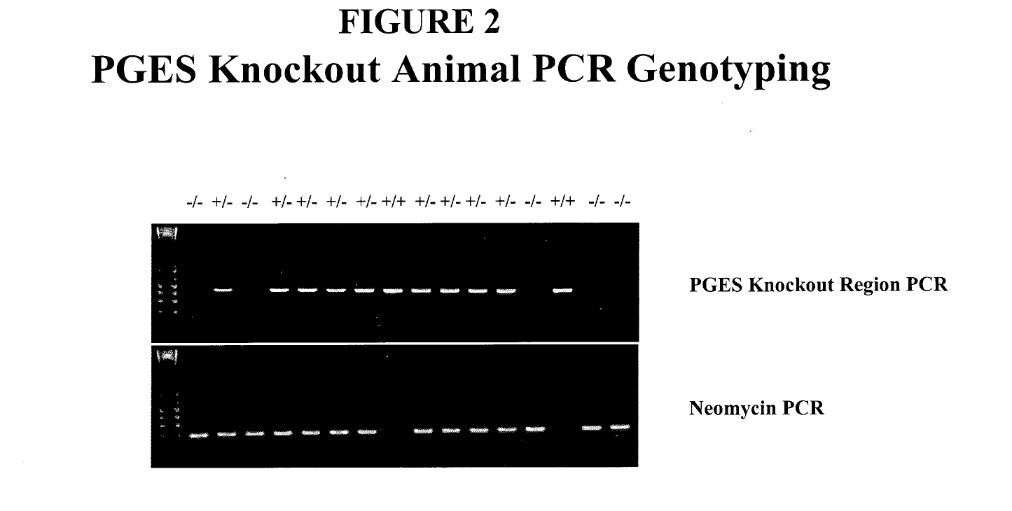

Thus, in determining genotype, a wild type animal would be positive for the PGES2 PCR and negative for the Neo PCR. A PGES2 heterozygous animal would be positive for both PCR reactions and a PGES2 KO animal would be negative for the PGES2 PCR (because the region is absent on both alleles) and positive for the Neo PCR (FIG. 2).

Initial histopathology of male and female wild type and PGES2 KO (n=1 for each sex and genotype) revealed no detectable differences. There were also no detectable differences between the fertility of the PGES2 KO mice and the wild type mice.

E. PGES2/ApoE Double KO Mice

PGES2/ApoE double KO mice were generated by crossing PGES2 KO mice to ApoE knockout mice (C57/BI6 background, Charles River Laboratories). PGES2+/−/ApoE −/− and PGES2−/−/ApoE−/− mice were generated.

F. PGES2 KO ES Cells

The DBA-252 murine ES cell line, derived from the DBA/1 LacJ cell line (Roach et al., Exp. Cell Res. 221:520-525, 1995) was used. Pluripotent ES cells were maintained in culture on a Mitomycin C treated primary embryonic fibroblast (PEF) feeder layer in stem cell medium (SCML) which contained the base medium Knockout D-MEM (ILTI, #10829-018) supplemented with 15% ES cell qualified fetal calf serum (ILTI, #10439-024), 0.1 mM 2-mercaptoethanol (Sigma Chemical, #M-7522), 0.2 mM L-glutamine (ILTI, #25030-081), 0.1 mM MEM non-essential amino acids (ILTI, #11140-050), 1000 u/ml recombinant murine leukemia inhibitory factor, and 50 μg/ml Gentamycin (ILTI, #15710-064). Electroporation of 1×10⁷ DBA-252 ES cells in 400 μl SCML with 25 μg linearized PGES2 KO targeting vector, as discussed in Example B, was carried out using a BTX Electro Cell Manipulator 600 (BTX, Inc.) at a voltage of 260 V, a capacitance of 50 μF, and a resistance of 360 Ohms. Following electroporation the cells were plated in SCML in four 100 mm tissue culture dishes on Mitomycin C treated PEFs. Twenty-four hours after electroporation, positive/negative selection was initiated by adding 175 μg/ml G418 and 2 μM gancyclovir to the SCML. Homologous recombination of the targeting vector into the ES cell genome deleted the mouse PGES2 gene and inserted the neomycin resistance gene. G418 resistant colonies were picked with a drawn micropipette into individual wells of a 24-well tissue culture dish following 7-9 days of G418 selection and expanded into clonal ES cell lines. Transformed ES cell lines that demonstrated gene targeting by homologous recombination were identified by Southern analysis.

PGES2 targeted (+/−) ES cell clones #22 and #70 were used for creating PGES KO (−/−) ES cells. The clones were thawed and maintained on PEFs in 175 μg/ml G418 in SCML for 2 days then the G418 concentration was increased to 2 mg/ml (Mortensen et al., Mol. Cell. Biol. 12:2391-95, 1992). After 7-10 days in high G418 selection, the surviving ES cell colonies were dissociated and 2-5×10⁵ cells/ml were plated onto new PEFs in 2 mg/ml G418 in SCML. After 4-7 additional days of high G418 selection, resistant colonies were picked with a drawn micropipette and transferred into individual wells of a 24-well tissue culture dish without PEFs in 2 mg/ml G418 in SCML and expanded into clonal ES cell lines. PGES2 KO (−/−) ES cell lines that demonstrated loss of the wild type allele were identified by Southern analysis.

G. PGES2 KO ES Cell-Derived Macrophages

DBA-252 WT (+/+) and PGES KO (−/−) ES cell clones #22D, #22F and #70V were used to develop ES cell in vitro differentiated (IVD) macrophages. WT and KO PGES2 ES cell clones were maintained without PEF feeders in SCML. Two days prior to embryoid body (EB) formation, all ES cell lines were switched to l-SCML medium that contained a base medium of Iscove's MDM (ILTI, #31980-030) supplemented with 15% ES cell qualified fetal calf serum, 0.2 mM L-glutamine, 0.1 mM MEM non-essential amino acids, 1000 u/ml recombinant murine leukemia inhibitory factor, 50 μg/ml Gentamycin and 0.1 mM 2-Mercaptoethanol (Sigma #M-7522).

Embryoid body stage: The WT and KO ES cell clones were dissociated and grown in suspension culture in bacteriology 100 mm dishes in MacEB medium that contained the base medium Iscove's MDM supplemented with 15% ES cell qualified fetal calf serum, 2 mM L-glutamine, 300 μg transferrin (ILTI, #13008-016), 50 μg/ml L-ascorbic acid (Sigma Chemical, #A-4403), 5% PFHM-II (ILTI, #12040-093), 4×10⁻⁴ M monothioglycerol (MTG), and 50 μg/ml Gentamycin. ES cells were grown in suspension for 6 days to form the EB cell aggregates.

Precursor macrophage stage: The EBs were dissociated on day 6 and plated in tissue culture dishes in Mac I medium that contained the base medium Iscove's MDM supplemented with 10% FBS (ILTI, #10439-024), 5% PFHM-II (ILTI, #12040-093), 2 mM L-glutamine, 3 ng/ml M-CSF (Sigma #M-9170), 1 ng/ml IL-3 (PeproTech, Inc., Rocky Hill, N.J., #213-13) and 50 μg/ml Gentamycin. When this cell population became confluent, the macrophage precursors developed as non-adherent clusters and could be harvested every other day from day 14 through day 30.

ES cell-derived macrophages: Non-adherent clusters of macrophage precursors were harvested from the media by centrifugation. Cell pellets were resuspended in Mac II media that contained the base medium Iscove's MDM supplemented with 10% FBS, 5% PFHM-II, 2 mM L-glutamine, 3 ng/ml M-CSF, and 50 μg/ml Gentamycin. Cells were plated onto tissue culture dishes or multi-well dishes and cultured for 1-5 days prior to characterization.

Rescue of wild type phenotype in the PGES2 KO ES cell-derived macrophages: It was observed that ES cell IVD macrophages derived from PGES2 KO ES cells clone #22F showed a decrease in viability and growth characteristics compared to DBA-252 WT and PGES2+/− clone #22 ES cell-derived macrophages. To determine if the phenotype was the result of the loss of PGE2 production, PGE2 (Cayman Chemicals #14010) was added to the media at all stages of the differentiation process at doses of either 0.1, 1.0 or 10 μM.

Macrophages from PGES2 KO clone #22F, cultured without PGE2 were approximately half the density of wild type. Macrophages from PGES2 KO clone #22F, cultured in 0.1 or 1.0 μM PGE2, were equivalent in density to wild type, and macrophages from PGES2 KO clone 22F, cultured in 10.0 μM PGE2, had a density of approximately 75% of wild type.

H. Characterization of PGES2 KO ES Cell-Derived Macrophages

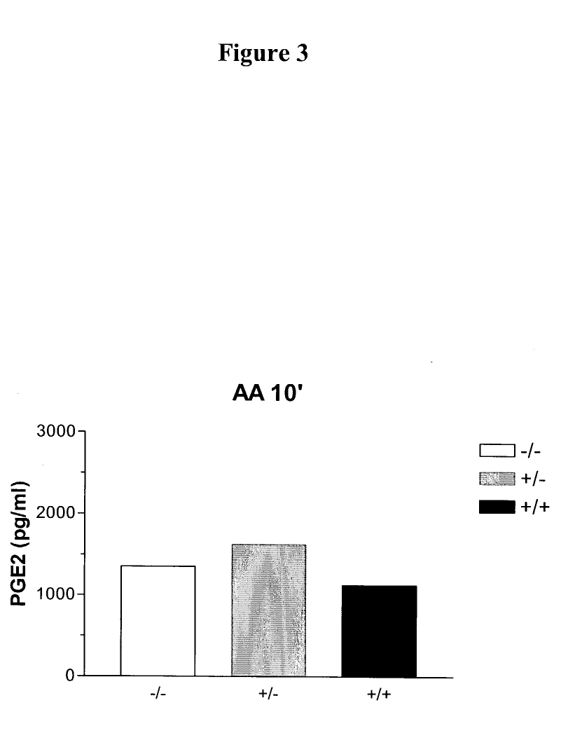

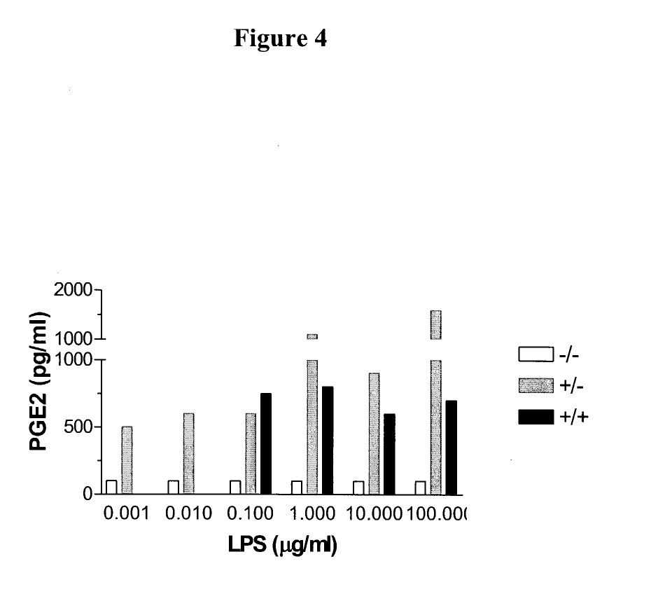

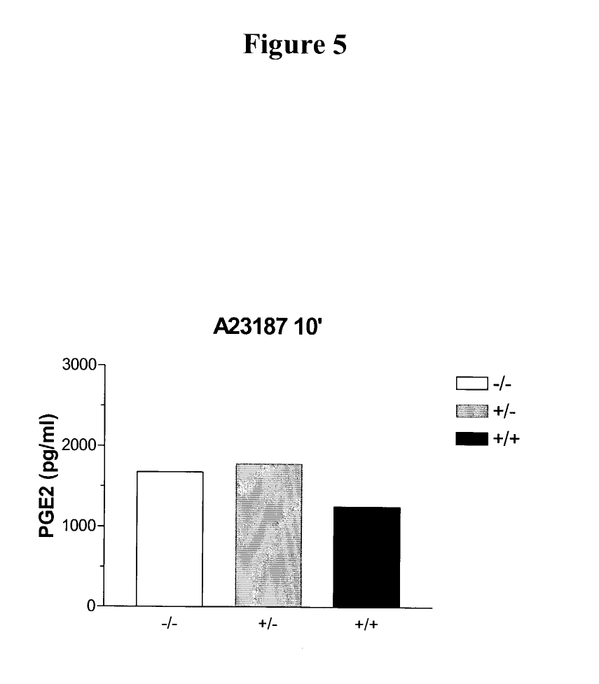

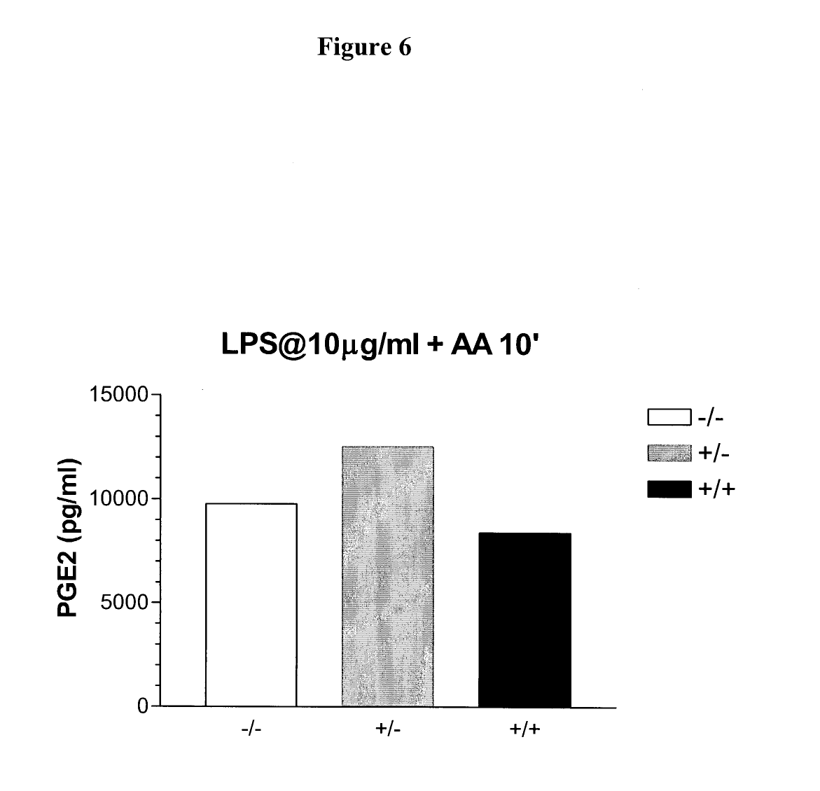

ES cell IVD macrophages (ESMs) derived from PGES2 KO, PGES2 heterozygote, and wild type ES cells, at day 14-21 of differentiation, were plated at 5×10⁵ cells/well (96 well-plate) in Mac II (see Example G) media. Cells were grown at 37° C. (5% CO2, 95% humidity) overnight and then stimulated under varying conditions: 1) the ESMs were incubated for 24 hours in the presence of the indicated concentrations of lipopolysaccharide (LPS) (FIG. 3) (a 1 mg/ml stock solution of LPS (E. coli 0111:B4, Sigma Chemical), in phosphate buffered saline (PBS), was diluted in cell media to achieve the desired final concentration ranging from 0.001-100 pg/ml); 2) the ESMs were incubated for 10 minutes with 100 μM arachidonic acid (AA) (Cayman Chemical Company, Ann Arbor, Mich.) (stock solution of 10 mM AA in 100% ethanol diluted in cell media to achieve the final concentration) (FIG. 4); 3) the ESMs were incubated for 10 minutes with the calcium ionophore A23187 (Calbiochem, San Diego, Calif.) (10 μM in 100% DMSO) (FIG. 5); and 4) the ESMs were incubated for 24 hours in the presence of 10 μg/ml LPS in cell media followed by a 10-minute incubation with 100 μM AA in cell media (FIG. 6).

At the end of each incubation period, cell supernatants were isolated and stored at −20° C. until assayed for PGE2. PGE2 measurements were performed with an ELISA detection kit (Cayman Chemical). All samples were diluted to yield a signal within the dynamic range of the standard curve. The data shown in each of FIGS. 3-6 are representative of three independent experiments, each experiment performed in duplicate.

As shown in FIG. 3, the results demonstrate that PGES2 is the pivotal PGE2 synthase responsible for the release of extracellular PGE2 under inflammatory conditions (e.g., with LPS stimulation). However, as shown in FIGS. 4, 5, and 6, the results also indicate that disruption of this PGES2 gene does not prohibit the ESMs from releasing PGE2 under more acute stimulation conditions (e.g., with arachidonic acid or calcium ionophore (A23187) stimulation). These results indicate that PGES2 is an important gene for the production of PGE2 during inflammation.

I. Characterization of PGES2 KO in Models of Inflammation

PGES2 KO mice were profiled in two experimental models of inflammation, collagen-induced arthritis (chronic inflammation model) and acetic acid-induced writhing (acute inflammation/pain model). All experiments were performed with age/sex-matched animals propagated on a DBA1/lacJ genetic background. This genetic background is optimal for collagen-induced arthritis (CIA). The phenotype in CIA was further profiled by characterizing the immune responses of PGES2 KO and wild type animals by assessing antibody production and delayed-type hypersensitivity reactions. In summary, results of the CIA and acetic acid-induced writhing models indicate a role for PGES2 in PGE2-mediated chronic inflammation, acute inflammation, and acute inflammatory pain detection as opposed to neuropathic pain detection.

Collagen-Induced Arthritis

Mice were immunized on day 0 with a solution of collagen and complete Freud's adjuvant prepared using the following reagents: acetic acid (glacial) (Sigma Chemical, #33,882-6), chick type II collagen (Chondrex, Redmond, Wash., # 20001-1), Mycobacterium tuberculosis H37RA (Becton Dickinson, Franklin Lakes, N.J., #231,141), and Freud's incomplete adjuvant (Sigma Chemical, #F-5506). 0.1M acetic acid was placed in an −80° C. freezer for 10 minutes until the solution became slushy. An aliquot of this acetic acid solution (5 ml) was then added to the 10 mg chick collagen vial (2 mg/ml), which was then wrapped in foil and placed at 4° C. for rocking overnight. Subsequently, 20 ml of Freud's incomplete adjuvant was placed in a glass tissue grinder (50 ml) and 40 mg of M. tuberculosis (2 mg/ml) was added and mixed until a uniform suspension was observed (i.e., complete Freud's adjuvant). Equal volumes of each solution were mixed for approximately 15 minutes until difficult to mix. All mice were then shaved at the base of their tails. Each animal was given an injection of 0.1 ml subcutaneously at the base of their tail on days 0 and 20.

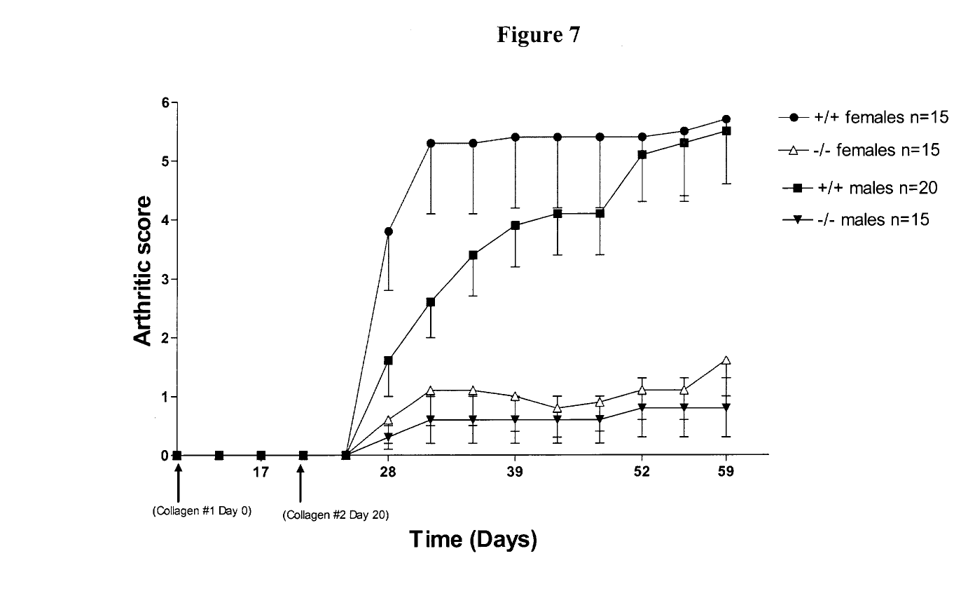

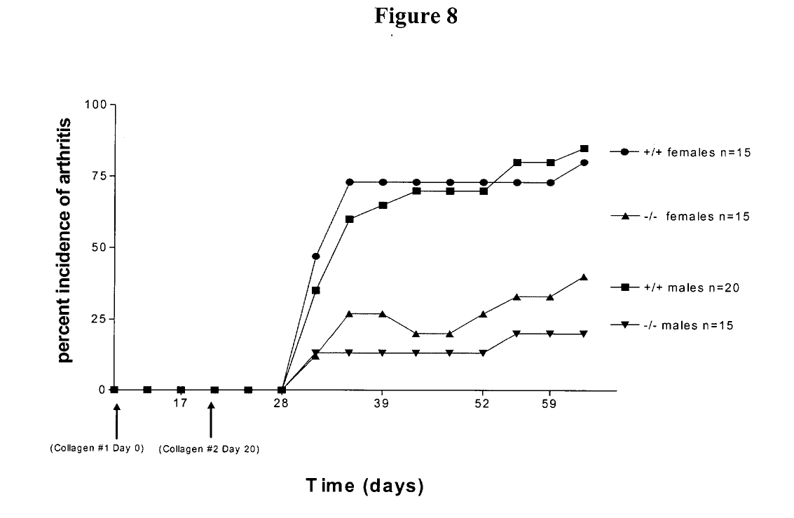

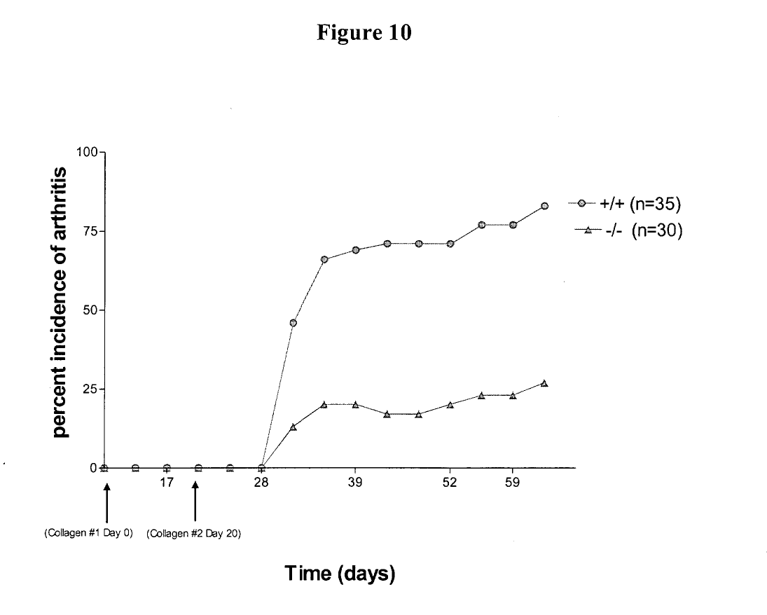

A second immunization was performed on day 20. By day 25, mice started developing the first signs of arthritis (red swollen joints). By day 50, the average arthritis score had reached its maximum. As shown in FIGS. 7-10, the incidence and severity of arthritis was attenuated in the PGES2 KO mice. Wild type controls reached a maximum severity with an arthritis score of 5.5±0.7 compared to PGES2 KO animals, reaching only a score of 1.1±0.4 on day 56 (FIGS. 7 and 9). Incidence was also significantly attenuated in the PGES2 KO group (FIGS. 8 and 10). In addition, histological examination revealed the absence of proteoglycan loss at articular surfaces of the collagen-treated PGES2 KO joints as compared to wild type.

To determine if the difference in arthritis was a result of deficient antibody production in PGES2 KO animals, antibody levels against type 11 collagen (the antigen) were determined by ELISA, using a mouse IgG type 11 collagen antibody ELISA kit (Chondrex, Redmond, Wash., #2031), goat anti-mouse IgG-HRP (Southern Biotech, Birmingham, Ala., #1031-05), goat anti-mouse IgGl-HRP (Southern Biotech, #1070-05), goat anti-mouse IgG2a-HRP (Southern Biotech, #1080-05), and goat anti-mouse IgG2b-HRP (Southern Biotech, #1090-05).

The instructions supplied with the kit were followed, except for the secondary step. Instead of using the lyophilized anti-IgG antibodies, HRP-conjugated isotype-specific antibodies that were diluted in the secondary dilution buffer (solution C of the Chondrex kit) were used.

Both wild type and PGES2 KO animals generated significant levels of IgG1, IgG2a and total IgG antibodies against type II collagen. There was no detectable difference in antibody production between mice from the two genotypes suggesting that the difference in arthritis was not due to an inability of PGES2 KO animals to mount an immune response against this particular antigen.

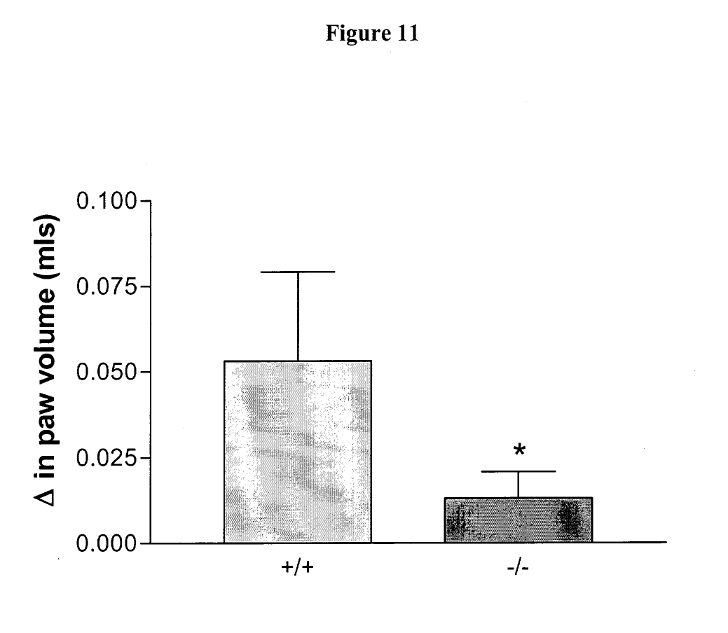

To determine the mechanism underlying the difference in severity and incidence of arthritis between wild type and PGES2 KO mice, delayed type hypersensitivity (DTH) responses were elicited in animals receiving a similar collagen immunization protocol. Mice were injected on day 0 at the base of their tail. On day 17, 10 μg (15 μl) of collagen was injected into the dorsal region of the right paw. The left paw from each animal was used as a control and received 15 μl of saline in the same location. On day 18, paw thickness was determined using a plethysmometer. Wild type mice developed significantly more swelling in collagen-treated paws versus contralateral saline-treated paws. The edema was associated with infiltration of white blood cells as determined by histopathological analysis. Edema formation in collagen-treated paws from PGES2 KO mice was similar to that of saline-treated paws from wild type or PGES2 KO animals (FIG. 11). This deficit was accompanied by a significant reduction in the number of white blood cells infiltrating the injection site, which is consistent with the role of PGES2 in inflammation.

In addition, blood isolated from PGES2 KO and wild type mice was analyzed for neutrophil, lymphocyte, monocyte, basophil and eosinophil counts. No detectable differences were observed between healthy and diseased animals indicating that a gross immunological defect did not cause the PGES2 KO phenotype observed in the CIA model.

Acetic Acid-Induced Writhing

Mice were randomized and dosed orally with either vehicle (0.5% (w/w) methylcellulose, Sigma Chemical, #M0512) or 10 mg/kg piroxicam (Sigma Chemical, #P5654). One hour later, 16 μl/g body weight of 0.7% acetic acid was administered intraperitoneally. The mice were placed in a 5-compartment box and the number of stretches was counted for 20 minutes following acetic acid injection.

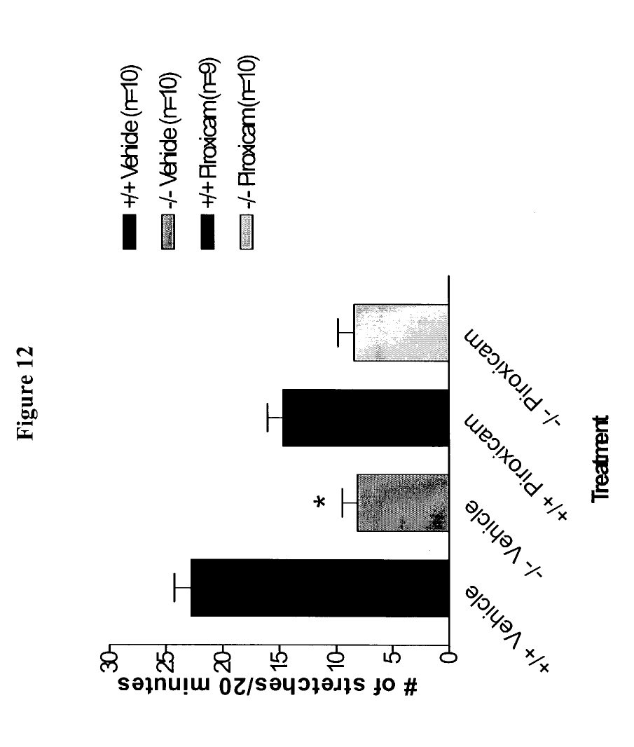

PGES2 KO mice demonstrated a reduced pain response as compared to the wild type mice (FIG. 12). Treatment with piroxicam reduced the response in wild type mice but had no effect on PGES2 KO mice.

The in vivo levels if the inflammatory prostaglandins 6-keto PGF1a (stable metabolite of PGI2) and PGE2 were also characterized. Injection of the acetic acid solution caused a significant elevation above baseline levels of both prostaglandins. No detectable differences in 6-keto PGF1a levels were recorded in either genotype regardless of treatment. By contrast, PGE2 levels were reduced by 52% in the PGES2 KO group as compared to wild type animals, consistent with the attenuated writhing response observed in PGES2 KO animals.

To further characterize the ability of PGES2 inhibition/disruption to modulate pain responses such as those at the spinal and supraspinal levels, withdrawal latencies were measured in PGES2 KO and wild type animals in the hot plate assay. No differences in responses were measured at 52.5, 55.5 or 58.5° C.

1. A genetically-modified, non-human mammal, wherein the modification results in a disrupted PGES2 gene.

2. The mammal of claim 1, wherein said mammal is a mouse.

3. The mammal of claim 1, wherein said mammal further comprises a disrupted ApoE gene.

4. A genetically-modified animal cell, wherein the modification comprises a disrupted PGES2 gene.

5. The animal cell of claim 4, wherein said cell is an embryonic stem (ES) cell or an ES-like cell.

6. The animal cell of claim 4, wherein said cell is an ES cell-derived macrophage.

7. The animal cell of claim 4, wherein said cell is isolated from a genetically-modified, non-human mammal containing a modification that results in a disrupted PGES2 gene.

8. The animal cell of claim 4, wherein said cell is utilized in culture media supplemented with PGE2.


SEQUENCE LISTING (4 sequences)

SEQ ID NO: 1 - DNA, 23 bp, Mus Musculus
gcaggatctcctgtcatctcacc

SEQ ID NO: 2 - DNA, 23 bp, Mus Musculus
gatgctcttcgtccagatcatcc

SEQ ID NO: 3 - DNA, 23 bp, Mus Musculus
tcccaggtgttgggatttagacg

SEQ ID NO: 4 - DNA, 23 bp, Mus Musculus
taggtggctgtactgtttgttgc